(19) United States
(12) Patent Application Publication
(10) Pub. No.: US 2009/0012425 A1
(43) Pub. Date: Jan. 8, 2009

(54) Apparatus and Method for Collecting a Sample of Material

(76) Inventors: Larry H. Dodge, Hernando, FL (US); Patrick A. Mach, Shorewood, MN (US)

Correspondence Address: 3M INNOVATIVE PROPERTIES COMPANY, PO BOX 33427, ST. PAUL, MN, 55133-3427, US

(21) Appl. No.: 11/997,575

(22) PCT Filed: Aug. 2, 2006
(86) PCT No.: PCT/US2006/030065

§ 371 Date: Jul. 31, 2008

Related U.S. Application Data
(60) Provisional application No. 60/705,140, filed on Aug. 2, 2005.

(51) Int. Cl. A61B 10/02

(52) U.S. Cl. 600/572; 600/573

(57) ABSTRACT

A sample acquisition device includes a hollow shaft and a porous medium. The hollow shaft includes a proximal end, a distal end, and a sidewall extending between the proximal end and the distal end. The sidewall of the hollow shaft includes a plurality of openings adjacent the distal end of the hollow shaft. The porous medium is attached to the distal end of the hollow shaft and at least a part of the porous medium is positioned over the plurality of openings.

### CROSS REFERENCE TO RELATED APPLICATIONS

This application claims the benefit of U.S. Provisional Patent Application No. 60/705,140, filed Aug. 2, 2005, which is incorporated herein by reference.

### BACKGROUND

Sample acquisition devices, such as swabs, are generally used in many industries for collecting a sample of material from a sample source. The sample acquisition device may include a hollow shaft including a distal end and a proximal end, and a porous medium attached to the distal end of the hollow shaft. Typically, the distal end and proximal end are open or include an opening. In the medical industry, the sample acquisition device may be used to gather a sample of biological material from a nose, ear, throat, or other sample source (e.g., a wound). Specifically, the hollow shaft may be handled to position the porous medium in contact with the nose, ear, throat, or other sample source. In the food service industry, the hollow shaft of the sample acquisition device may be handled to position the porous medium in contact with a food preparation surface, a food container, and the like. The samples collected by the sample acquisition device may then be analyzed for the presence of an organism (an “analyte”). The analysis may incorporate an assay.

Prior to the analysis of the sample, the sample is typically transferred from the porous medium in order to place the sample in condition for analysis. In some methods, the porous medium may be placed in contact with a slide or other laboratory apparatus in order to transfer at least some of the sample from the porous medium to the slide or other laboratory apparatus. In other methods, a fluid, such as a buffer solution, may be introduced into the proximal end of the hollow shaft of the sample acquisition device. The fluid then flows through the hollow shaft and exits through an opening at the distal end and contacts the porous medium.

### BRIEF SUMMARY

The application discloses, in one aspect, a sample acquisition device comprising a hollow shaft and a porous medium. The hollow shaft includes a proximal end, a distal end, and a sidewall extending between the proximal end and the distal end. In the embodiments disclosed, the sidewall includes a plurality of openings proximate to the distal end. The porous medium is attached to the hollow shaft wherein at least a part of the porous medium is positioned over the plurality of sidewall openings.

A method is disclosed of collecting a sample of material. The method comprises contacting a porous medium of a sample acquisition device with a sample source, introducing a fluid into a proximal end of a hollow shaft of the sample acquisition device, and eluting a sample from the sample acquisition device via fluid flow through a plurality of openings along a side wall of the hollow shaft.

The above summary is not intended to describe each disclosed embodiment or every implementation of the present invention. The figures and the detailed description which follow more particularly exemplify illustrative embodiments.

### BRIEF DESCRIPTION OF THE DRAWINGS

The present invention will be further explained with reference to the drawing figures listed below, where like structure is referenced by like numerals throughout the several views.

FIG. 1 is a side view of a sample acquisition device of the prior art.

FIG. 2A is a side view of an exemplary embodiment of a sample acquisition device of the present invention, which includes a hollow shaft comprising a plurality of radial openings adjacent a distal end of the hollow shaft, and a porous medium positioned over the plurality of radial openings.

FIG. 2B is a schematic cross-sectional view of the sample acquisition device of FIG. 2A.

FIG. 3 is a schematic cross-sectional view of a sample acquisition assembly of the present invention, which includes the sample acquisition device of FIGS. 2A and 2B attached to a syringe and enclosed by a housing, where the housing includes an outlet port.

While the above-identified figures set forth an exemplary embodiment of the present invention, other embodiments are also within the invention. In all cases, this disclosure presents the invention by way of representation and not limitation. It should be understood that numerous other modifications and embodiments can be devised by those skilled in the art, which fall within the scope and spirit of the principles of the invention.

### DETAILED DESCRIPTION

The present invention is a sample acquisition device including a hollow shaft comprising a sidewall that includes a plurality of openings, and a porous medium attached to the hollow shaft, where at least a part of the porous medium is positioned over the plurality of openings. In the present invention, a plurality of radial openings are positioned along a sidewall of the hollow shaft of the sample acquisition device. In some embodiments, the hollow shaft includes a distal opening as well as a plurality of radial openings along the sidewall. The hollow shaft includes a proximal end, a distal end, and the sidewall extends between the proximal end and the distal end. The plurality of openings in the sidewall are adjacent the distal end of the hollow shaft. In some embodiments, the plurality of openings are uniform in size, while in other embodiments, at least two of the openings in the plurality of openings are different sizes. The proximal end of the hollow shaft includes an opening (a “proximal opening”), and in some embodiments, the distal end of the hollow shaft includes at least one opening (a “distal opening”). In those embodiments where the hollow shaft includes a distal opening, the porous medium is positioned over the distal opening.

The hollow shaft of the inventive sample acquisition device may be formed of any suitable material, including polycarbonate, or other similar plastics. For example, an injection molding process utilizing a plastic material may be used to form the hollow shaft. The porous medium may be any suitable porous medium, such as a fibrous bud comprised of rayon fibers, Dacron fibers, or combinations thereof. While the present invention is described in reference to a fibrous bud, other porous media are contemplated and those skilled in the art may substitute a suitable porous medium into the exemplary sample acquisition device described herein. For example, in other embodiments, the porous medium may include bristles, rather than a fibrous bud.

The inventive sample acquisition device may be used to collect a sample of material from a sample source, such as a nose, ear, or throat of a person, or a food preparation surface. Specifically, a technician may handle the hollow shaft of the device to contact the porous medium with a sample source. The sample is then collected in or on (or “adhered to”) the porous medium of the sample acquisition device. Subsequent to collecting the sample, the sample may be analyzed to detect the presence of an organism. As described in the Background section, in some circumstances, the sample must be eluted from the porous medium. The present invention provides a novel sample acquisition device that, in some cases, may improve the percent of the sample eluted (i.e., the percent recovery) from the porous medium.

The inventive sample acquisition device includes openings along a sidewall of a hollow shaft of the device, where at least a part of the porous medium is positioned over the openings. A fluid is typically introduced into the proximal end of the hollow shaft in order to elute at least some of the sample from the porous medium. The openings on the sidewall of the hollow shaft allow the fluid to exit along a side portion of the porous medium, and thus help elute the sample that is collected along the side portion of the porous medium as well as the sample that is collected along the end portion of the porous medium. The side portion is generally the portion that contacts the sidewall of the hollow shaft, while the end portion of the porous medium is the portion that contacts the distal end of the hollow shaft. When the fluid exits an opening along the sidewall of the hollow shaft, it may help elute more sample from the side portion as compared to a sample acquisition device configured to allow the fluid to exit only at the distal end. This may be advantageous if a sample is collected on the side portion of the porous medium. The elution is described in further detail in reference to the figures.

In some embodiments, an inventive sample acquisition device is incorporated into a sample acquisition assembly, which includes the sample acquisition device and a fluid reservoir retaining a fluid. The fluid reservoir may be attached to the proximal end of the hollow shaft of the sample acquisition device, or the reservoir may be manually positioned at the proximal end of the hollow shaft by a sample acquisition device technician (i.e., any person using the device). The fluid reservoir is preferably in selective fluidic communication with the hollow shaft of the sample acquisition device. “Selective fluidic communication” indicates that there is a valve, plunger (such as in a syringe) or other technician-activated means of introducing a fluid disposed in the fluid reservoir into the hollow shaft of the sample acquisition device. Otherwise stated, it is preferred that a technician is able to control the dispensing of a fluid contained in a fluid reservoir in a sample acquisition assembly of the present invention.

The fluid reservoir may be any suitable reservoir including a means for selectively releasing the contents of the reservoir. For example, the fluid reservoir may be a deformable squeeze bulb that is manually squeezed by a technician, such as described in U.S. Pat. No. 5,266,255, entitled, “SPECIMEN TEST UNIT”, or a syringe-type reservoir. As it will be later described, the selection of the type of fluid reservoir may depend upon the amount of release pressure that is needed or desired. “Release pressure” is the pressure with which the fluid is released from the fluid reservoir.

After a sample is acquired in or on the porous medium of the sample acquisition device, the fluid retained in the fluid reservoir may be introduced into the hollow shaft to elute at least some of the sample from the porous medium. Specifically, the fluid moves through the hollow shaft and contacts the porous medium and in a sense, “flushes” at least some of the sample from the porous medium. In order for the fluid to elute at least some of the sample from the porous medium, at least some of the fluid must contact the porous medium. For this reason, it is important for the hollow shaft of the sample acquisition device to include at least one opening. In prior art sample acquisition devices, an opening is positioned at the distal end of the hollow shaft of the sample acquisition device.

In sample acquisition devices containing one distal opening, it is likely that the fluid does not contact the side portion of the hollow shaft under the porous medium as much as the fluid contacts the end portion. As a result, the amount of sample that is eluted from the side may be less than the amount of sample that is eluted from the end portion. This may be problematic in certain applications because a sufficient quantity of the sample may not be eluted from the porous medium, which may then affect the accuracy of the sample analysis. This is especially true if a large quantity of the sample is adhered to side portion of the porous medium (e.g., as a result of the technique with which the sample was collected).

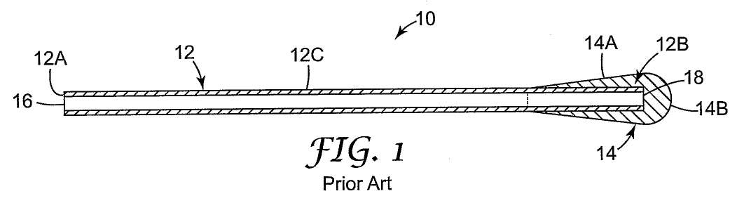

FIG. 1 illustrates a sample acquisition device 10 embodiment of the prior art. As shown, the sample acquisition device 10 includes a hollow shaft 12 having a sidewall 12C. Hollow shaft 12 has an elongate length that extends between a proximal end 12A and a distal end 12B having openings 16 and 18, respectively. As shown, a porous medium 14 is attached to the distal end 12B of the hollow shaft 12 and covers distal opening 18. A sample is collected on the porous medium 14. Fluid is introduced into a channel of the hollow shaft 12 through opening 16. Fluid flows through the channel and out opening 18. As shown, the porous medium 14 includes a side portion 14A and an end portion 14B

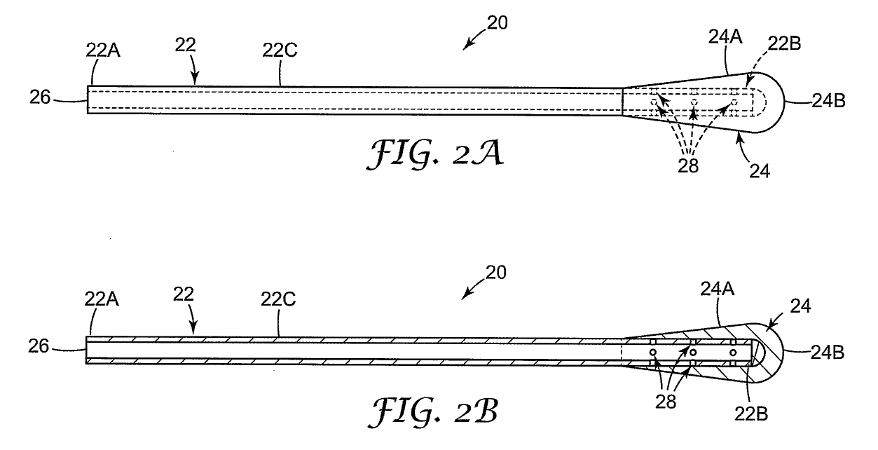

FIGS. 2A and 2B illustrate an embodiment of a sample acquisition device 20 according to the present invention. FIG. 2A is a side view of an exemplary embodiment of a sample acquisition device 20 of the present invention. FIG. 2B is a schematic cross-sectional view of sample acquisition device 20 of FIG. 2A. Sample acquisition device 20 includes a hollow shaft 22 with a proximal end 22A, a distal end 22B (shown in phantom) opposite proximal end 22A, and a sidewall 22C extending between a proximal end 22A and a distal end 22B. A porous medium 24 is attached proximate to the distal end 22B. In some embodiments, the porous medium 24 is a fibrous bud. Hollow shaft 22 is cylindrical and includes proximal opening 26 at proximal end 22A. In the exemplary embodiment, distal end 22B does not include a distal opening. However, in alternate embodiments, distal end 22B may include a single distal opening, or a plurality of openings.

In one embodiment, sidewall 22C is round in lateral section, and includes a plurality of radial openings 28 (shown in phantom) adjacent distal end 22B of hollow shaft 22. Openings 28 are radial in the sense that openings 28 are positioned about sidewall 22C and equidistant from a centerline through a center of hollow shaft 22. Fibrous bud 24 is positioned over the plurality of radial openings 28 and is attached to hollow shaft 22 using any suitable means, such as winding the fiber around hollow shaft 22. At least a portion of fibrous bud 24 may be formed of any suitable material, such as natural and synthetic polymeric materials, including but not limited to cellulosic, polyester, rayon, polyurethane or combinations thereof.

In the exemplary embodiment, openings in plurality of openings 28 are equal in size. In alternate embodiments, at least two openings in plurality of openings 28 may be different sizes. For example, in one embodiment, radial openings 28 may be graded, where radial openings 28 closest to proximal end 22A are larger than radial openings 28 closest to distal end 22B, or vice versa. Openings 28 of various sizes may help to increase or decrease fluid pressure through openings 28 and/or otherwise control fluid flow through openings 28. Those skilled in the art may modify the size of openings 28 accordingly. Further, even though openings 28 are shown to be round, in alternate embodiments, openings 28 may be any suitable shape.

A technician may use hollow shaft 22 as a handle to contact fibrous bud 24 with a sample source. After a sample is adhered to fibrous bud 24, the sample is eluted from fibrous bud 24 in order to analyze it. If a method of eluting the sample from fibrous bud 24 is used, a fluid is introduced into proximal opening 26. Thereafter, the fluid flows through hollow shaft 22, and exits through the plurality of radial openings 28. Because the plurality of openings 28 are positioned along sidewall 22C of hollow shaft 22, the fluid will exit through sidewall 22C of hollow shaft 22, and contact a side portion 24A of fibrous bud 24. The probability that the fluid will contact side portion 24A of fibrous bud 24 and elute the sample of material adhered to side portion 24A is increased. Openings 28 also allow the fluid to contact end portion 24B of fibrous bud 24. This may result in a higher sample elution percentage in certain cases. For this reason as well as others, it may be advantageous to use inventive sample acquisition device 20 for samples that are mainly acquired on side portion 24A of fibrous bud 24.

A sample acquisition device of the present invention may be introduced into a sample processing apparatus, such as the ones described in U.S. Patent Application Ser. No. 60/705,088, entitled “Apparatus and Method for Detecting an Analyte” (Attorney Docket No. 61044US002), U.S. Patent Application Ser. No. 60/705,118, entitled “Apparatus Assembly and Method for Detecting an Analyte” (Attorney Docket No. 61114US002), U.S. Patent Application Ser. No. 60/705,089, entitled “Apparatus and Method for Detecting an Analyte” (Attorney Docket No. 61095US002) and U.S. Patent Application Ser. No. 60/705,090, entitled “Apparatus and Method for Detecting an Analyte” (Attorney Docket No. 61106US002) all filed on Aug. 2, 2005.

Table 1 illustrates how a higher percent of sample may be eluted from the inventive sample acquisition device 20. Specifically, Table 1 shows the results of an experiment in which a controlled amount of sample was adhered to a porous medium of a sample acquisition device. The sample was comprised of Staphylococcus aureus, (ATCC 25923, Rockville, Md.) and PBS-L64 buffer solution. PBS-L64 buffer solution is a phosphate buffer solution including 0.20 Moles of NaPO₄ and 0.15 Moles of NaCl with a pH of 7.5 containing 0.2% (weight/volume) of PLURONIC L64 Surfactant (made commercially available by BASF Corporation, Mount Olive, N.J.). PBS-L64 buffer solution was then introduced into a proximal end of a hollow shaft of a sample acquisition device using a syringe. Column 2 shows the average (among 10 trials) percent recovery of the sample from a sample acquisition device with only a distal opening and the standard deviation among the ten trials. Column 3 shows the average (among 10 trials) percent recovery of the sample from a sample acquisition device with a plurality of radial openings and the standard deviation among the ten trials.

TABLE 1
Recovery of Staphylococcus Aureus from a Sample Acquisition Device by
Elution with Buffer (Average of 10 Trials)

| | Swab Description: | |
|---|---|---|
| | | Hollow Shaft With |
| | Hollow Shaft With Only | a Plurality of |
| | a Distal Opening | Radial Openings |
| Recovery (%) | 59 | 113 |
| Standard Deviation | 29 | 38 |
| (%) | | |

As Table 1 illustrates, the percent (%) recovery with a sample acquisition device including a hollow shaft with radial openings (e.g., sample acquisition device 20 of FIGS. 2A and 2B) is 113%, almost twice the percent recovery (59%) with a sample acquisition device including a hollow shaft with only a distal opening (e.g., sample acquisition device 10 of FIG. 1). The sample acquisition device including a hollow shaft with radial openings has greater than 100% recovery because staphylococcus aureus is an organism that clumps together, and after the staphylococcus aureus was eluted from the porous medium of the sample acquisition device, at least some of the clumps broke apart after the elution, resulting in more clumps than the sample originally contained.

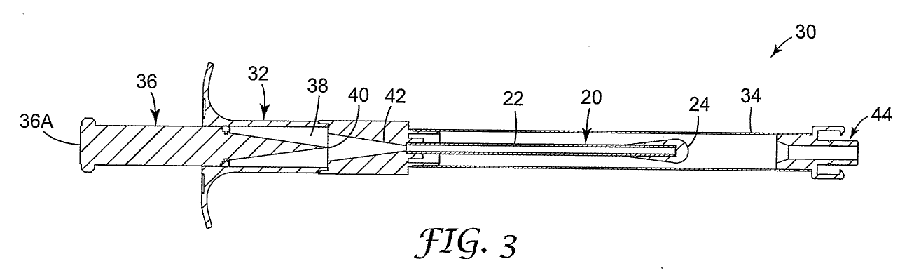

FIG. 3 is a schematic cross-sectional view of sample acquisition assembly 30 of the present invention, which includes sample acquisition device 20 of FIGS. 2A and 2B, syringe 32, and housing 34, which encloses sample acquisition device 20. Sample acquisition device 20 and syringe 32 may be preattached (e.g., at the manufacturing level), or a technician may attach the same in the field (e.g., at the site of the sample source). Sample acquisition device 20 and syringe 32 are attached using a Luer fitting, such as a Luer slip fitting or a Luer lock fitting (not shown). However, in alternate embodiments, any suitable means of attaching sample acquisition device 20 and syringe 32 may be used. Syringe 32 and housing 34 are attached using any suitable means, including a mechanical mating system.

Syringe 32 may be any suitable syringe known in the art. In the exemplary embodiment, syringe 32 includes plunger member 36, chamber 38, and frangible membrane 40. Chamber 38 may be prefilled with a fluid, such as a buffer solution, for eluting a sample from a porous medium or fibrous bud 24 of sample acquisition device 20. In alternate embodiments, a technician may fill chamber 38 with a fluid. Examples of buffer solutions that may be used include PLURONIC L64 Surfactant, made commercially available by BASF Corporation, Mount Olive, N.J., and the commercial RAMP diluent made commercially available by Response BioMedical Corp., Burnaby, British Columbia, Canada. The type of buffer solution retained in chamber 38 may depend upon the type of testing process used and the type of analyte sought to be detected.

Frangible membrane 40 forms a part of chamber 38 and helps to ensure that the fluid retained in chamber 38 is not prematurely (i.e., before purposeful activation of syringe 32 by a technician) released into hollow shaft 22 of sample acquisition device 20. For example, frangible member 38 may be an aluminum seal. In alternate embodiments, syringe 32 may include other means for preventing syringe 32 from prematurely activating, or a means for locking syringe 32 so that plunger member 36 may not be activated until purposeful activation by a technician. With a preattached syringe, a locking device may be necessary in order to help prevent the fluid retained in chamber 38 from contaminating the sample source during sample collection.

Plunger member 36 is configured to break through frangible membrane 40 in order to release the fluid retained in chamber 38. Specifically, a technician may press down on a top portion 36A of plunger member 36 to depress plunger member 36 into recess 42. In this way, chamber 38 is in selective fluidic communication with hollow shaft of sample acquisition device 20. Recess 42 is configured to receive plunger member 36. In the exemplary embodiment of sample acquisition assembly 30, recess 42 and plunger member 36 share a similar cross-section and mate together. A first state of plunger member 36 is shown in FIG. 3. In a second state, after plunger member 36 is depressed, plunger member 36 is in close conforming contact with recess 42.

Housing 34 is formed of any suitable material, including a blow-molded plastic. Housing 34 is configured to fit over sample acquisition device 20 and interfit with syringe 32 (such as by mechanically mating components). Sample acquisition device 20 or assembly 30 of the present invention may be provided to a consumer as a sterile unit. This may be a desirable feature for medical applications. Housing 34 may help to maintain the sterility of sample acquisition device 20.

Housing 34 may include a filter or other capture medium to isolate an analyte from the sample of material eluted from fibrous bud 24 of sample acquisition device 20. Housing 34 includes outlet port 44, which may be, for example, a drip tip. Outlet port 44 is configured to be coupled with a sample processing apparatus such as the ones described in U.S. Patent Application Ser. No. 60/705,088, entitled “Apparatus and Method for Detecting an Analyte” (Attorney Docket No. 61044US002), U.S. Patent Application Ser. No. 60/705,118, entitled “Apparatus Assembly and Method for Detecting an Analyte” (Attorney Docket No. 61114US002), U.S. Patent Application Ser. No. 60/705,089, entitled “Apparatus and Method for Detecting an Analyte” (Attorney Docket No. 61095US002) and U.S. Patent Application Ser. No. 60/705,090, entitled “Apparatus and Method for Detecting an Analyte” (Attorney Docket No. 61106US002) all filed on Aug. 2, 2005.

After a sample is acquired with sample acquisition device 20, housing 34 may be positioned to generally enclose sample acquisition device 20. A fluid contained in chamber 38 of syringe 32 may then be released by depressing plunger member 36. The fluid is then introduced into hollow shaft 22 of sample acquisition device 20 and contacts fibrous bud 24, thereby eluting at least some of the sample of material from fibrous bud 24. The fluid and sample then gather in housing 34 and exit housing 34 through outlet port 44. In some embodiments the fluid and sample may pass through a capture medium disposed in housing 34 prior to exiting through outlet port 44. If outlet port 44 is coupled with a sample processing apparatus, the fluid and sample are introduced into the sample processing apparatus through outlet port 44.

While sample acquisition assembly 30 includes syringe 32 as a fluid reservoir, alternate embodiments of inventive sample acquisition assemblies may include any suitable fluid reservoir. For example, the fluid reservoir may be a deformable squeeze bulb or an accordion pleat bulb. Syringe 32 or its equivalents may be beneficial for processes that are testing for the presence of certain organisms that require a high quantity for detection. In order to increase the accuracy of the testing process, it may be desirable to elute as much of the sample from fibrous bud 24 as possible. Syringe 32 releases a fluid with higher pressure than, for example, a deformable squeeze bulb. A higher pressure in addition to radial openings 28 may help elute more of the sample from fibrous bud 24.

In alternate embodiments, housing 34 may be eliminated from sample acquisition assembly 30 and sample acquisition device 20 may be directly introduced into a sample-processing device, such as those described in U.S. Patent Application Ser. No. 60/705,088, entitled “Apparatus and Method for Detecting an Analyte” (Attorney Docket No. 61044US002), U.S. Patent Application Ser. No. 60/705,118, entitled “Apparatus Assembly and Method for Detecting an Analyte” (Attorney Docket No. 61114US002), and U.S. Patent Application Ser. No. 60/705,089, entitled “Apparatus and Method for Detecting an Analyte” (Attorney Docket No. 61095US002) all filed on Aug. 2, 2005.

During an analyte detection process, the analyte (whether it is isolated from the sample of material or not) is typically exposed to a reagent. The reagent may be dehydrated in order to keep the reagent in a stable form during storage of the sample acquisition device or assembly. In the present invention, a dehydrated reagent may be disposed in the sample acquisition device or the sample acquisition assembly. For example, in the sample acquisition device, the reagent may be disposed in the hollow shaft (e.g., hollow shaft 22 of FIGS. 2A and 2B). In the sample acquisition assembly, the reagent may be disposed in the syringe (e.g., in recess 42 of syringe 32), in the housing (e.g., housing 34 shown in FIG. 3), or in the outlet port (e.g., outlet port 44 shown in FIG. 3).

Although the present invention has been described with reference to preferred embodiments, workers skilled in the art will recognize that changes may be made in form and detail without departing from the spirit and scope of the invention.

The complete disclosures of the patents, patent documents and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. A sample acquisition device comprising:
a hollow shaft comprising:
a proximal end;
a distal end; and
a sidewall extending between the proximal end and the distal end, the sidewall comprising a plurality of openings proximate to the distal end wherein at least two openings of the plurality of openings in the sidewall are different sizes; and
a porous medium attached to the hollow shaft, wherein at least a part of the porous medium is positioned over the plurality of openings.

2. The sample acquisition device of claim 1, wherein the distal end of the hollow shaft comprises a distal opening, and wherein the porous medium is positioned over the distal opening.

3. The sample acquisition device of claim 1 wherein the porous medium is a fibrous bud.

4. The sample acquisition device of claim 3 wherein the fibrous bud comprises a material selected from a group consisting of rayon fibers, Dacron fibers, and combinations of rayon fibers and Dacron fibers.

5. (canceled)

6. The sample acquisition device of claim 1 and further comprising:
a fluid reservoir attached to the proximal end of the hollow shaft and in selective fluidic communication therewith.

7. The sample acquisition device of claim 6 wherein the fluid reservoir is a syringe comprising:
a chamber for retaining a fluid; and
a plunger member moveable between a first state and a second state to express fluid.

8. The sample acquisition device of claim 7, wherein the syringe is attached to the proximal end of the hollow shaft using an attachment selected from a group consisting of a Luer slip fitting and a Luer lock fitting.

9. The sample acquisition device of claim 7, wherein the fluid reservoir comprises a buffer solution.

10. The sample acquisition device of claim 1 in combination with
a syringe positioned in selective fluidic communication with the proximal end of the hollow shaft of the sample acquisition device.

11. The sample acquisition assembly of claim 1 and further comprising a housing surrounding the sample acquisition device.

12. A method of collecting a sample of material, the method comprising:
contacting a porous medium of a sample acquisition device with a sample source;
introducing a fluid into a proximal end of a hollow shaft of the sample acquisition device; and
eluting a sample from the sample acquisition device via fluid flow through a plurality of openings along a side wall of the hollow shaft.

13. The method of claim 12, wherein the fluid is released from a fluid reservoir.

14. The method of claim 12, wherein the step of introducing the fluid comprises:
moving a plunger of a syringe from a first position to a second position to release the fluid from a chamber into the proximal end of the hollow shaft.

15. The method of claim 14, wherein prior to releasing the fluid contained in the chamber, the method comprises:
attaching the syringe to the proximal end of the hollow shaft of the sample acquisition device.

16. The method of claim 12, and further comprising the step of:
eluting the sample from the sample acquisition device via fluid flow through a distal opening of the sample acquisition device.
